(19) United States
(12) Patent Application Publication
(10) Pub. No.: US 2024/0017049 A1
(43) Pub. Date: Jan. 18, 2024

(54) Blood Draw Connector for Vascular Access System

(71) Applicant: Becton, Dickinson and Company, Franklin Lakes, NJ (US)

(72) Inventors: Deepan Vikash, Nagercoil K.K Dist (IN); Sakthivel Karthikeyan, Bangalore (IN); Jonathan Karl Burkholz, Salt Lake City, UT (US)

(21) Appl. No.: 18/222,022

(22) Filed: Jul. 14, 2023

(30) Foreign Application Priority Data
Jul. 15, 2022 (IN) 202211040724

(51) Int. Cl. A61M 39/02; A61M 25/00; A61M 25/01

(52) U.S. Cl. CPC A61M 39/02; A61M 25/0097; A61M 25/0113; A61M 2039/0258; A61M 2039/027; A61M 2039/0282

(57) ABSTRACT

A vascular access system configured for delivery of a tube or probe into a patient's vascular system, the vascular access system including a catheter adapter having a distal portion and a proximal portion, wherein a catheter extends from the distal portion and a septum assembly is housed within the proximal portion, and a connector assembly. The connector assembly includes a coupling interface configured to couple the connector assembly to the proximal portion of the catheter adapter, and a cannula. The cannula may be configured to extend through the septum assembly of the catheter adapter when the connector assembly is coupled to the proximal portion of the catheter adapter. The connector assembly also includes a proximal interface, wherein the proximal interface is configured for coupling to a blood draw device having an elongated core extending from a distal end thereof.

### CROSS-REFERENCE TO RELATED APPLICATION

This application claims priority to Indian Provisional Patent Application No. 202211040724 filed Jul. 15, 2022, the disclosure of which is hereby incorporated by reference in its entirety.

### BACKGROUND OF THE INVENTION

### Field of the Invention

The present disclosure generally relates to vascular access systems such as, e.g., peripheral intravenous catheters (PIVCs). More particularly, the present disclosure relates to vascular access systems having an add-on connector for improved compatibility with blood draw and/or probe delivery devices.

### Description of Related Art

A catheter is commonly used to infuse fluids into vasculature of a patient. For example, the catheter may be used for infusing normal saline solution, various medicaments, or total parenteral nutrition. Furthermore, the catheter may also be used for withdrawing blood from the patient.

The catheter may be an over-the-needle peripheral intravenous catheter (PIVC). In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. After proper placement of the needle, the clinician may temporarily occlude flow in the vasculature and remove the needle, leaving the catheter in place (i.e., “indwelled”) for future blood withdrawal and/or fluid infusion.

In order to complete blood draws from PIVCs having indwelled catheters, blood draw devices have been developed that are configured to overcome previous challenges related to blood draw through PIVCs such as, e.g., the possibility of catheter collapse, reduced blood flow due to debris built up on or within the catheter, etc. One such device, PIVO™ from Velano Vascular, Inc., is configured as a single-use device which temporarily attaches to a PIVC to draw a blood sample. Using an existing peripheral intravenous line as a conduit to the vasculature, the PIVO™ device advances a probe in the form of a flow tube through the PIVC, beyond the catheter tip, and into the patient's vein in order to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded.

In addition to blood draw devices such as the PIVO™ device described above, similar devices for advancing a probe (e.g., a nickel titanium wire, guidewire, instrument, obturator, rod, wire with fluid path, and/or sensor) through an indwelling catheter and into a patient's vasculature have also been developed. However, existing vascular access devices are not generally optimized for compatibility with these blood draw and/or probe delivery devices. For example, the tube or probe of such devices are typically configured to access the indwelling catheter via a side port of a catheter adapter of the PIVC, which may increase the risk of undesirable hemolysis and/or bending of the tube or probe due to the curved pathway of the side port. Additionally, a separate needle free connector (NFC) is generally needed to enable compatibility of existing vascular access devices with, e.g., the PIVO™ device.

### SUMMARY OF THE INVENTION

Accordingly, the present disclosure generally relates to a vascular access system configured for delivery of a tube or probe into a patient's vascular system, the vascular access system including a catheter adapter comprising a distal portion and a proximal portion, wherein a catheter extends from the distal portion and a septum assembly is housed within the proximal portion. The system also includes a connector assembly, wherein the connector assembly includes a coupling interface configured to couple the connector assembly to the proximal portion of the catheter adapter, a cannula, wherein the cannula is configured to extend through the septum assembly of the catheter adapter when the connector assembly is coupled to the proximal portion of the catheter adapter, and a proximal interface, wherein the proximal interface is configured for coupling to a blood draw device having an elongated core extending from a distal end thereof.

In certain configurations, the vascular access system includes at least a portion of the cannula that is slidable within the connector assembly. The connector assembly may also include a biasing member, wherein the biasing member is configured to bias the cannula proximally within the connector assembly. The biasing member may be a steel spring. Optionally, the biasing member is a rubber bellow.

In certain configurations, the cannula may be configured to at least partially extend through an opening formed in a distal end portion of the connector assembly. In some embodiments, the vascular access system further includes an O-ring seal surrounding the opening formed in the distal end portion of the connector assembly. Optionally, the vascular access system further includes a split septum positioned at a proximal end portion of the connector assembly. The cannula may include a proximal interface surface configured to receive at least a portion of a distal end of the elongated core of the blood draw device when the blood draw device is coupled to the connector assembly.

In other configurations, the coupling interface of the connector assembly includes a plurality of extensions projecting distally therefrom. The connector assembly may also include a groove formed in a sidewall thereof, wherein the groove is configured to receive a distal end of one or more clips of a coupler member of the blood draw device. The cannula may be fixed and extend distally from the coupling interface of the connector assembly. In certain configurations, the connector assembly may also include a slidable actuator disposed therein, wherein the actuator is configured to selectively pierce a split septum positioned within a body of the connector assembly.

In certain embodiments, the proximal interface of the connector assembly may include a needle-free connector. The proximal interface of the connector assembly may include a T-extension set.

In accordance with certain embodiments of the present invention, a connector assembly configured for use with a vascular access system for delivery of a tube or probe into a patient's vascular system includes a coupling interface configured to couple the connector assembly to a proximal portion of a catheter adapter of the vascular access system, and a cannula, wherein the cannula is configured to selectively extend through a septum assembly of the catheter adapter. The system may also include a proximal interface, wherein the proximal interface is configured for coupling to a blood draw device having an elongated core extending from a distal end thereof.

In certain configurations, at least a portion of the cannula is slidable within the connector assembly. Optionally, the system includes a biasing member configured to bias the cannula proximally within the connector assembly. The cannula may be fixed and extend distally from the coupling interface of the connector assembly. The connector assembly may also include a slidable actuator disposed therein, wherein the actuator is configured to selectively pierce a split septum positioned within a body of the connector assembly.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a perspective view of a vascular access system in accordance with an aspect of the present disclosure;

FIG. 2A is a perspective view of a connector assembly for use with the vascular access system of FIG. 1;

FIG. 2B is a side cross-sectional view of the connector assembly of FIG. 2A;

FIG. 2C is a perspective cross-sectional view of the connector assembly of FIG. 2A;

FIG. 3 is a partial perspective cross-sectional view of the vascular access system of FIG. 1;

FIG. 4A is a partial side cross-sectional view of the vascular access system of FIG. 1 in a first configuration;

FIG. 4B is a partial side cross-sectional view of the vascular access system of FIG. 1 in a second configuration;

FIG. 4C is a partial side cross-sectional view of the vascular access system of FIG. 1 in a third configuration;

FIG. 5 is a side cross-sectional view of a connector assembly in accordance with another aspect of the present disclosure;

FIG. 6A is a perspective view of a connector assembly in accordance with another aspect of the present disclosure;

FIG. 6B is a perspective cross-sectional view of the connector assembly of FIG. 6A;

FIG. 6C is an exploded view of the connector assembly of FIG. 6A;

FIG. 7A is a perspective view of a connector assembly in accordance with another aspect of the present disclosure;

FIG. 7B is a perspective cross-sectional view of the connector assembly of FIG. 7A;

FIG. 7C is a partial cross-sectional view of a blood draw device in accordance with another aspect of the present disclosure;

FIG. 8 is a perspective view of a connector assembly in accordance with another aspect of the present disclosure;

FIG. 9 is a perspective view of a connector assembly in accordance with another aspect of the present disclosure;

FIG. 10A is a perspective view of a connector assembly in accordance with another aspect of the present disclosure;

FIG. 10B is a perspective cross-sectional view of the connector assembly of FIG. 10A;

FIG. 10C is a perspective cross-sectional view of the connector assembly of FIG. 10A coupled to a blood draw device;

FIG. 11A is a perspective view of a connector assembly in accordance with another aspect of the present disclosure;

FIG. 11B is a side cross-sectional view of the connector assembly of FIG. 11A;

FIG. 11C is a side view of the connector assembly of FIG. 11A with an end cap;

FIG. 12A is a perspective view of a connector assembly in accordance with another aspect of the present disclosure;

FIG. 12B is a perspective cross-sectional view of the connector assembly of FIG. 12A coupled to a blood draw device; and

FIG. 13 is a perspective view of a connector assembly in accordance with another aspect of the present disclosure.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw or probe delivery device in preparation for or during use. Similarly, in this application, the terms “in the distal direction” and “distally” mean in the direction toward the connector portion of the fluid transfer device, and the terms “in the proximal direction” and “proximally” mean in the direction opposite the direction of the connector.

Embodiments of the present disclosure will primarily be described in the context of vascular access systems including an integrated peripheral IV catheter (PIVC). While not shown or described herein, it is to be understood that the connector assemblies described below may be utilized for blood draw and/or probe advancement through any suitable vascular access device such as, e.g., the BD NEXIVA™ Closed IV Catheter system. However, embodiments of the present disclosure equally extend to use with other catheter devices.

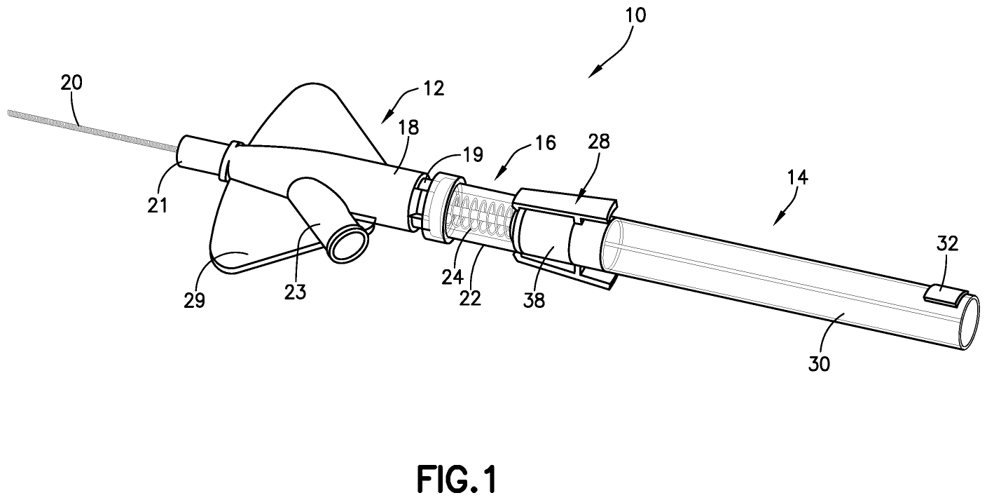

Referring to FIG. 1, a vascular access system 10 in accordance with an aspect of the present disclosure is illustrated. The vascular access system 10 includes an integrated catheter adapter 12, with the catheter adapter 12 having a catheter 20 extending distally from a distal end portion 21, wherein the catheter 20 is configured for insertion into a patient's vasculature. As will be described in detail hereinbelow, the vascular access system 10 further includes a connector assembly 16, wherein a distal end portion of the connector assembly 16 is configured to be couplable to a proximal portion 18 of the catheter adapter 12, while a proximal end portion of the connector assembly 16 is configured to be couplable to a blood draw device 14. The blood draw device 14 may be any appropriate device capable of advancing a tube or probe therefrom for the collection of a blood sample via the indwelling catheter 20. For example, in one embodiment, the blood draw device 14 may be a PIVO™ device from Velano Vascular, Inc. However, the blood draw device 14 in accordance with the present disclosure is not limited as such.

Referring still to FIG. 1, the catheter adapter 12 may further include a pair of wing members 29, wherein the pair of wing members 29 may, e.g., aid in deployment of the catheter 20, provide stabilization for the catheter adapter 12, provide greater surface area for dressing and securement of the catheter adapter 12, etc. A side port 23 may be provided, with side port 23 being in fluid communication with the catheter 20. While not shown, in some embodiments, an extension set may be coupled to the side port 23 so as to provide a fluid conduit for fluid infusion, flushing, etc.

As noted above, the blood draw device 14 is configured to selectively advance a tube or probe for the purpose of collecting a blood sample via the indwelling catheter 20. In the embodiment shown in FIG. 1, the blood draw device includes an elongated introducer body 30 having an actuator 32 slidably disposed thereon. The actuator 32 is operably coupled to the tube or probe (not shown) such that distal movement of the actuator 32 correspondingly advances the tube or probe through the blood draw device 14, through the connector assembly 16, and through the catheter adapter 12 until a distal tip of the tube or probe reaches to or beyond a distal tip of the catheter 20 so as to enable the tube or probe to act as a conduit for blood collection into a blood collection container (not shown) fluidly coupled thereto. The blood draw device 14 may further include a coupler member 28 for coupling the blood draw device 14 to a proximal portion 38 of the connector assembly 16. In some embodiments, the coupler member 28 may be configured as an alligator-clip member. However, it is to be understood that coupler member 28 may be configured as any interface or device capable of coupling the blood draw device 14 to the connector assembly 16.

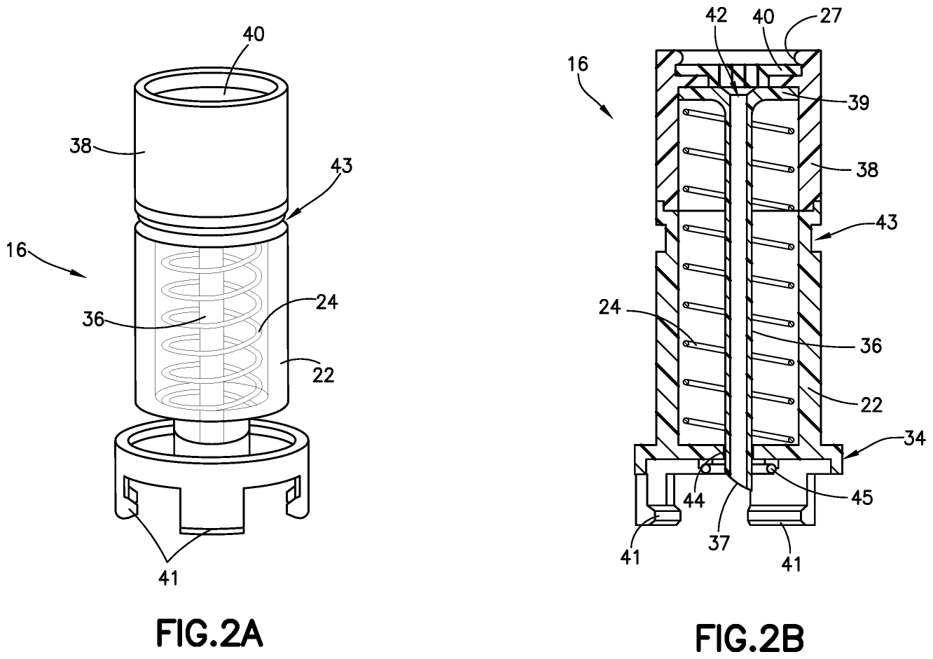

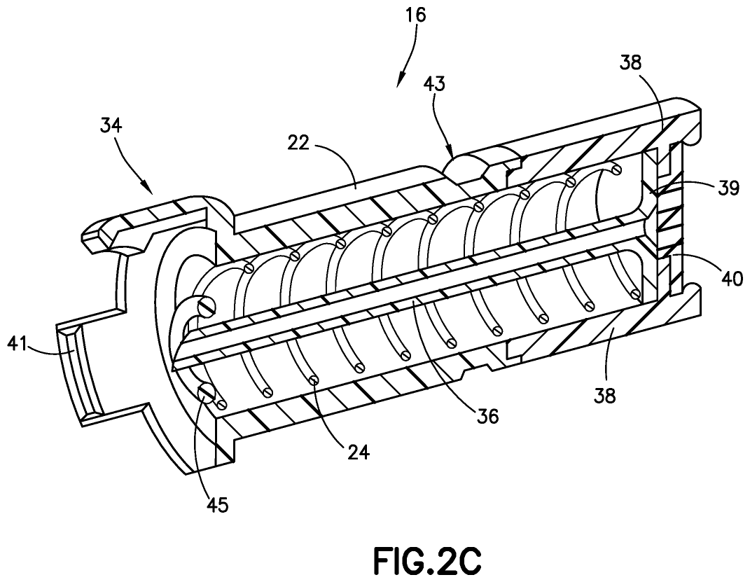

Referring now to FIGS. 2A-2C, the connector assembly 16 in accordance with an aspect of the present disclosure is shown in further detail. Connector assembly 16 may include a primary housing 22 and a secondary housing 38, which may be coupled together by any appropriate method such as, e.g., welding, adhesive, threads, etc. However, in alternative embodiments, it is to be understood that primary housing 22 and secondary housing 38 may be formed as a single member. Connector assembly 16 further comprises a distal coupling interface 34 extending from the primary housing 22. In some embodiments, distal coupling interface 34 may include a plurality of extensions 41, wherein the extensions 41 are configured to snap into a groove or recess 19 formed on an outer surface of the proximal portion 18 of the catheter adapter 12, thereby securing the connector assembly 16 to the catheter adapter 12, as well as preventing rotation of the connector assembly 16 with respect to the catheter adapter 12. However, it is to be understood that distal coupling interface 34 may utilize any appropriate coupling method for securing the connector assembly 16 to the catheter adapter 12, and is not limited to the features shown and described herein.

The primary housing 22 and secondary housing 38 are configured to slidably retain a cannula 36 therein, wherein the cannula 36 is selectively movable along an interior of the connector assembly 16. The cannula 36 extends distally from a bevel portion 39 and includes a distal point 37 capable of piercing, e.g., a septum of a catheter adapter. The cannula 36 may be formed of any appropriate material such as, e.g., plastic, metal, etc.

A biasing member such as, e.g., a spring 34 is also provided within the interior of the connector assembly 16. In a first configuration, the spring 24 is configured to bias the cannula 36 and bevel portion 39 in a proximal direction such that the cannula 36 is substantially retained within the connector assembly 16, with only a small portion of cannula 36 and distal tip 37 extending from an opening 44 formed near the distal coupling interface 34, as is shown in FIGS. 2B and 2C. However, as will be described in further detail below, in a second configuration, the spring 24 may be compressed such that a substantial portion of the cannula 36 extends distally from the connector assembly 16, thereby enabling the cannula 36 to extend through the septum of a catheter adapter.

In some embodiments, the bevel portion 39 may include one or more vent openings (not shown) formed therein, with the one or more vent openings configured to reduce drag forces on the cannula 36 and bevel portion 39 due to air resistance during slidable movement within the connector assembly 16. Additionally, an O-ring seal 45 may be positioned around (and distally to) the opening 44 so as to provide a fluid-tight seal when the connector assembly 16 is coupled to a catheter adapter. The O-ring seal 45 may be formed of any appropriate material such as, e.g., rubber.

Referring still to FIGS. 2A-2C, the connector assembly 16 further includes a split septum 40 positioned near a proximal end thereof, with the split septum 40 configured to selectively seal the interior of the connector assembly 16, while providing selective access to a proximal interface surface 42 of the cannula 36 when a blood draw device is coupled to the connector assembly 16, as will be described further hereinbelow. In some embodiments, the split septum 40 may be retained within the secondary housing 38 by an annular projection 27 formed at a proximal end of the secondary housing 38. However, it is to be understood that split septum may be retained via any suitable method and is not limited to use with the annular projection 27 as shown in FIG. 2B.

In some embodiments, the connector assembly 16 may also include at least one groove 43 formed in a sidewall thereof. The groove 43 may be sized and configured to receive, e.g., the distal end of one or more clips of the coupler member 28 for coupling the blood draw device 14 to a proximal portion 26 of the connector assembly 16, as is shown in FIG. 1. In some embodiments, the size and/or location of the groove 43 may be determined based on compatibility with a specific blood draw device such as, e.g., the PIVO™ device described above.

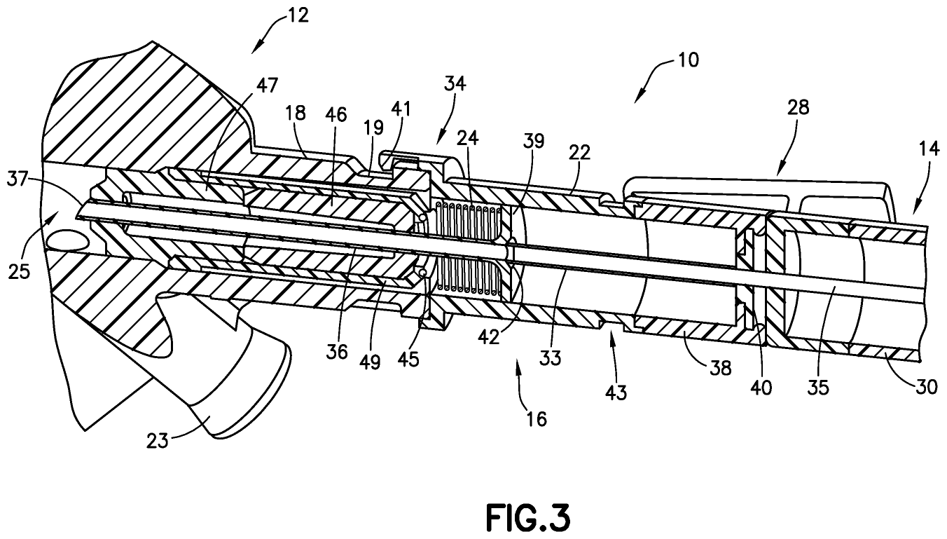

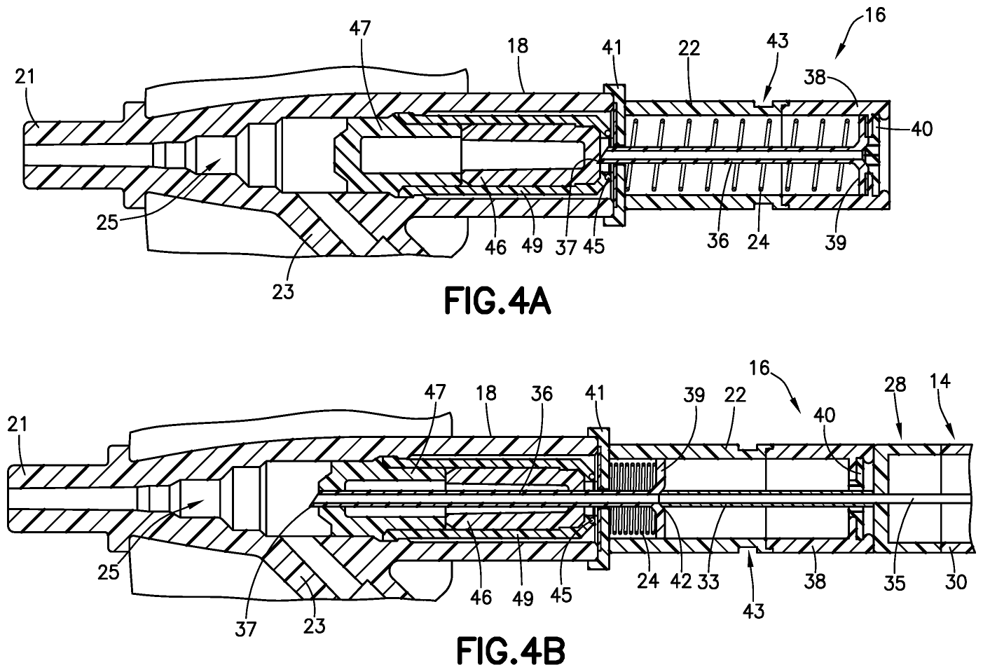

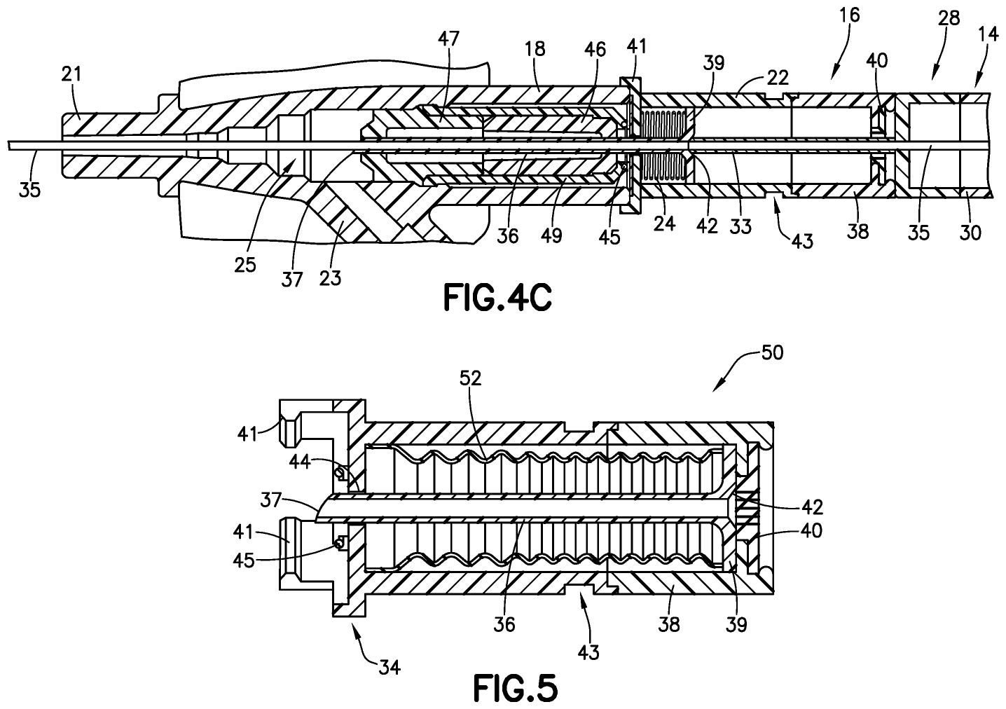

Referring now to FIGS. 3 and 4A-4C, vascular access system 10 in various configurations in accordance with an aspect of the present disclosure is illustrated. First, referring to FIG. 4A, the connector assembly 16 is initially coupled to the proximal portion 18 of the catheter adapter 12. Within the proximal portion 18, the catheter adapter 12 includes an internal canister 49, with canister 49 configured to retain both a primary septum 46 and a secondary septum 47, with the combined primary septum 46 and secondary septum 47 acting to form a fluid seal for the proximal portion 18 of catheter adapter 12 when not pierced by a needle or cannula, thereby preventing fluid passage to or from an interior conduit 25 of catheter adapter 12 past the primary septum 46 and/or the secondary septum 47. While FIGS. 3 and 4A-4C show a two-piece primary septum 46 and secondary septum 47 being used, it is to be understood that a single-piece (or more than two-piece) septum may be used in accordance with another aspect of the present disclosure.

As is shown in FIG. 4A, when the connector assembly 16 is initially coupled to the proximal portion 18 of the catheter adapter 12, the O-ring seal 45 contacts a proximal end of the canister 49 to form a substantially fluid-tight seal between the connector assembly 16 and an interior of the proximal portion 18. However, in this initial configuration, the cannula 36 is in an “idle” position, with the spring 24 acting to bias the cannula 36 proximally within the connector assembly 16 such that the distal tip 37 of cannula 36 does not pierce or otherwise penetrate the primary septum 46, thereby maintaining fluid isolation between the interior conduit 25 of the catheter adapter 12 and the cannula 36.

However, referring to FIG. 3 and FIG. 4B, when the blood draw device 14 is coupled to the connector assembly 16, an elongated core 33 extending from the distal end of blood draw device 14 is configured to penetrate the split septum 40 of the connector assembly 16, with a blunt distal tip of the elongated core 33 configured to contact the proximal interface surface 42 of the cannula 36 so as to form a fluid passageway from the cannula 36 into the blood draw device 14. As the elongated core 33 moves distally during coupling of the blood draw device 14 to the connector assembly 16, the bevel portion 39 and cannula 36 are correspondingly pushed distally, thereby compressing the spring 24 and allowing the cannula 36 to pierce the primary septum 46 and the secondary septum 47, thereby providing a fluid pathway between the interior conduit 25 of the catheter adapter 12 and the cannula 36. In some embodiments, the proximal interface surface 42 may be configured so as to enable mating with blood draw devices and/or probe-advancement devices having elongated cores of varying diameters.

With the fluid pathway between the blood draw device 14 and the catheter adapter 12 established via the cannula 36, a clinician may utilize the blood draw device 14 to advance a blood draw tube 35 through the elongated core 33, the cannula 36, the interior conduit 25, and into and/or beyond the indwelling catheter (not shown) extending from the catheter adapter 12, as is shown in FIG. 4C. With the blood draw tube 35 in an advanced position within the patient's vasculature, the clinician may collect one or more blood samples via the blood draw device 14. Once a blood draw procedure is complete, the clinician may decouple the blood draw device 14 from the connector assembly 16, thereby withdrawing the elongated core 33 from the connector assembly 16 and allowing spring 24 to bias the cannula 36 to the idle state shown in FIG. 4A. The connector assembly 16 may remain in this idle state until another blood draw device and/or other probe-advancement device is coupled thereto. Accordingly, the same connector assembly 16 is capable of being activated multiple times to provide for multiple blood draw procedures from the indwelling catheter.

Using the connector assembly 16 shown and described above with respect to FIGS. 1-4C, a blood draw device may be fluidly coupled to the proximal portion 18 of the catheter adapter 12, which is substantially in-line with the indwelling catheter 20. This in-line configuration enables the blood draw tube of the blood draw device to enter the patient's vasculature along a substantially straight path, thereby avoiding the curved entry path of prior art devices, which were configured to enter via, e.g., the side port 23. With the blood draw tube extending along this substantially straight path, the risk of kinking of the tube and/or hemolysis of the blood sample is reduced. Additionally, the use of connector assembly 16 does not require any alterations to the existing catheter adapter 12, blood draw device 14, and/or any extension sets coupled thereto.

Next, referring to FIG. 5, a connector assembly 50 in accordance with another aspect of the present disclosure is shown. While connector assembly 16 described above with respect to FIGS. 1-4C utilized a spring 24 to bias the cannula 36 proximally when in an idle state, connector assembly 50 uses a rubber bellow 52 to provide a similar biasing force on the bevel portion 39 of the cannula 36. It is to be understood that the present disclosure is not limited to use with a spring or rubber bellow, and any appropriate biasing means may be utilized within a connector assembly.

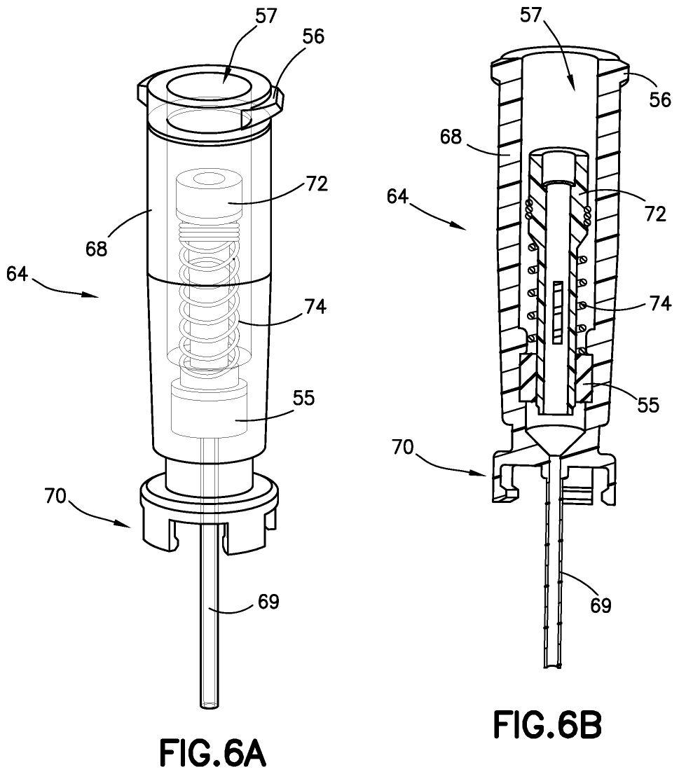

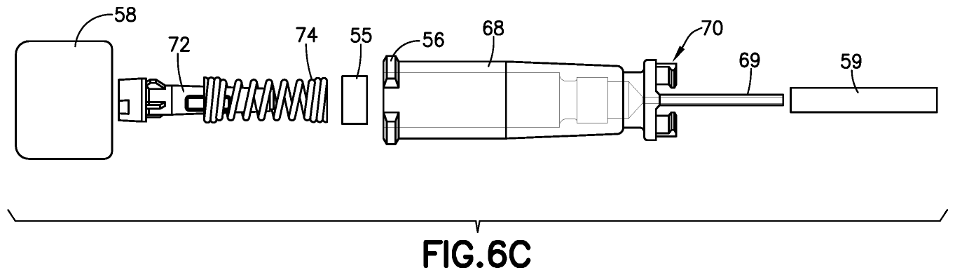

Referring now to FIGS. 6A-6C, a connector assembly 64 in accordance with another aspect of the present disclosure is shown. Unlike connector assembly 16 described above, which includes a slidable cannula 36 that is configured to selectively pierce a septum portion of a catheter adapter 12 to provide a fluid connection between the connector assembly 16 and the catheter adapter 12, connector assembly 64 includes a fixed cannula 69 extending from a distal end portion of a body 68, wherein the fixed cannula 69 is configured to penetrate a septum portion of a catheter adapter (not shown) when the connector assembly 64 is coupled to a proximal portion of the catheter adapter. The connector assembly 64 includes a coupling interface 70 which is configured to secure the connector assembly to the proximal portion of the catheter adapter, similar to coupling interface 34 described above with respect to FIGS. 1-4C.

Connector assembly 64 further includes a proximal interface 56 configured to receive a coupling member of a blood draw device such that an elongated core of a blood draw device (not shown) is able to enter a conduit 57 of the connector assembly 64. In some embodiments, the proximal interface 56 is a luer connection.

Furthermore, an actuator member 72 is provided within the conduit 57, with the actuator member 72 being biased via a spring 74 in a proximal direction. A split septum 55 is positioned at a distal portion of the conduit 57, with the split septum 55 capable of being selectively opened and closed by a distal end portion of the actuator member 72. More specifically, when a blood draw device is coupled to the connector assembly 64, the elongated core of the blood draw device is configured to contact the actuator member 72, pressing the actuator member 72 against the force of spring 74 until a distal end portion of the actuator member 72 pierces the split septum 55. When the actuator member 72 fully pierces the split septum 55, the actuator member 72, having a channel formed therethrough, provides fluid communication between the fixed cannula 69 and the blood draw device (not shown) coupled to the connector assembly 64. A tube or probe of the blood draw device may then be advanced through the connector assembly 64 and the catheter adapter (not shown) to which it is attached so as to perform a blood draw procedure. When the blood draw procedure is completed, the blood draw device may be decoupled from the proximal interface 56 of the connector assembly 64, thereby retracting the elongated core of the blood draw device such that the spring 74 biases the actuator member 72 in a proximal direction to again close the split septum 55 and, thus, close fluid communication between the catheter adapter and the actuator member 72.

In some embodiments, the connector assembly 64 may include an end cap 58, which may be positioned over the proximal interface 56 when a blood draw device is not in use. Additionally and/or alternatively, a cannula cover 59 may also be provided to cover and protect the fixed cannula 69 prior to insertion into a catheter adapter. Furthermore, in some embodiments, the body 68 of connector assembly 64 may include one or more grooves formed thereon, with the one or more grooves being configured to accept, e.g., the distal ends of clips of a coupling member of a blood draw device.

Using the connector assembly 64 shown and described above with respect to FIGS. 6A-6C, a blood draw device may be fluidly coupled to the proximal portion of the catheter adapter, which is substantially in-line with the indwelling catheter. This in-line configuration enables the blood draw tube of the blood draw device to enter the patient's vasculature along a substantially straight path, thereby avoiding the curved entry path of prior art devices.

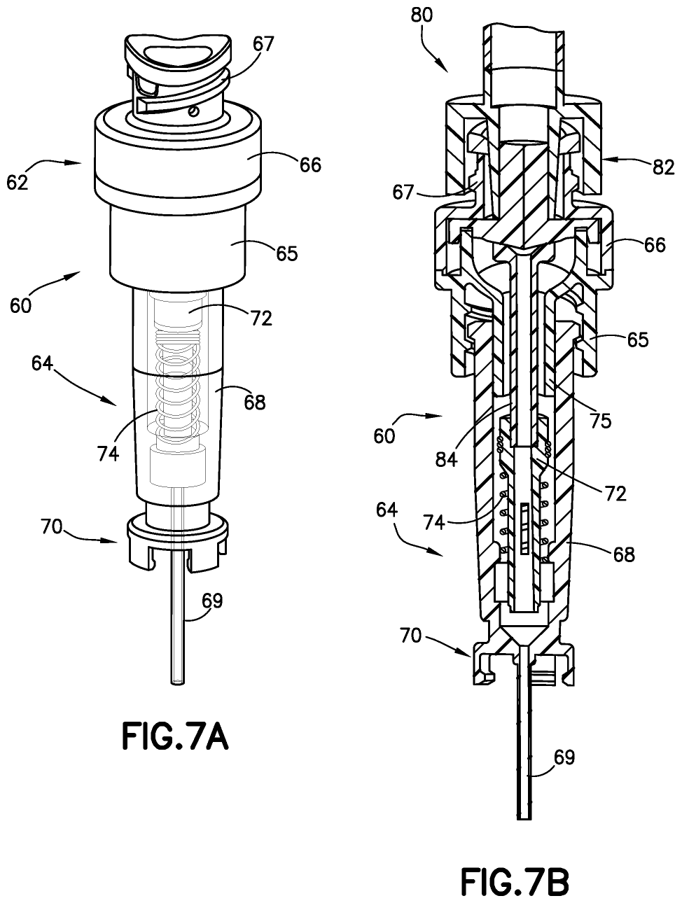

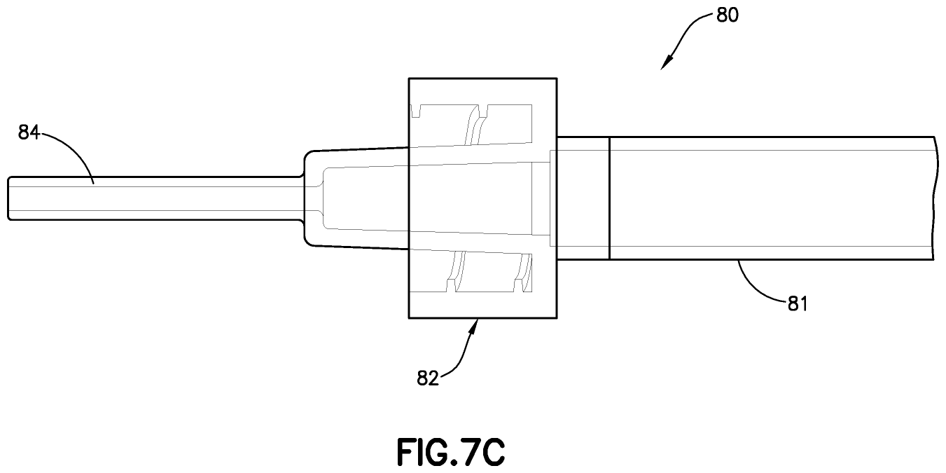

Next, referring to FIGS. 7A and 7B, a needle-free connector assembly 60 in accordance with another aspect of the present disclosure is shown. Needle-free connector assembly 60 includes the connector assembly 64 described above with respect to FIGS. 6A and 6B, but further includes a needle-free connector 62. In some embodiments, needle-free connector 62 may be configured as, e.g., a BD Q-Syte™ Needle-Free Connector from Becton, Dickinson and Co. The needle-free connector 62 may include a distal interface portion 65 extending distally from a body 66, and a proximal interface portion 67 extending proximally from the body 66. In some embodiments, the distal interface portion 65 may be configured as a male luer connector, while the proximal interface portion 67 may be configured as a female luer connector with a split septum seal formed therein.

Referring to FIGS. 7B and 7C, the needle-free connector assembly 60 is configured for use with a blood draw device 80. Unlike the blood draw devices described above with respect to FIGS. 1-6C, which were configured to be usable with a blood draw device having an alligator clip-type coupling member, blood draw device 80 includes a male luer lock coupling member 82, which is configured for coupling with the proximal interface portion 67. The blood draw device 80 further includes an introducer body 81 and an elongated core 84 extending distally from the male luer lock coupling member 82. As shown in FIG. 7B, when the blood draw device 80 is coupled to the needle-free connector assembly 60, the elongated core 84 is configured to open the split septum seal and pass through a male luer slip 75 of the distal interface portion 65 to contact and distally move the actuator 72 such that the blood draw device 80 is placed in fluid communication with the fixed cannula 69 and, thus, the catheter adapter (not shown).

Due to the presence of the needle-free connector 62, needle-free connector assembly 60 advantageously may be flushed via conventional flushing methods, and also provides an effective seal against blood reflux. Additionally, the needle-free connector 62 is disinfectable prior to initial use and between each use to aid in the prevention of sample contamination and/or the introduction of contaminants.

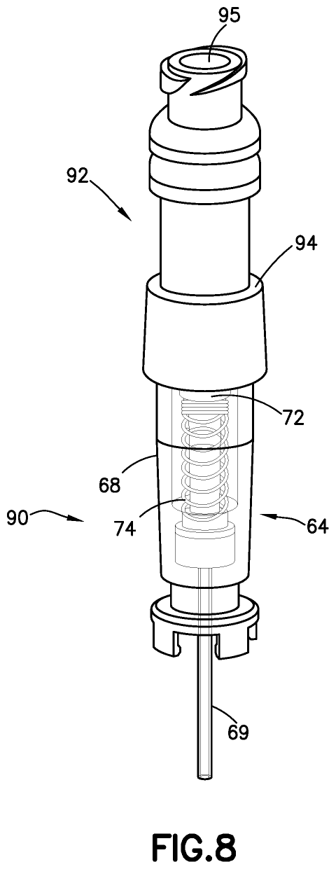

FIG. 8 illustrates a needle-free connector assembly 90 in accordance with another aspect of the present disclosure. Similar to needle-free connector assembly 60 described above with respect to FIGS. 7A and 7B, needle-free connector assembly 90 includes the connector assembly 64 described above with respect to FIGS. 6A and 6B, but further includes a needle-free connector 92. In some embodiments, needle-free connector 92 may be configured as, e.g., a BD SmartSite™ Needle-Free Connector from Becton, Dickinson and Co. The needle-free connector 92 may include a distal interface portion 94 and a proximal interface portion 95. As with needle-free connector assembly 60 described above, needle-free connector assembly 90 also may be flushed via conventional flushing methods, and provides an effective seal against blood reflux. Additionally, the needle-free connector 92 is disinfectable prior to initial use and between each use to aid in the prevention of sample contamination and/or the introduction of contaminants.

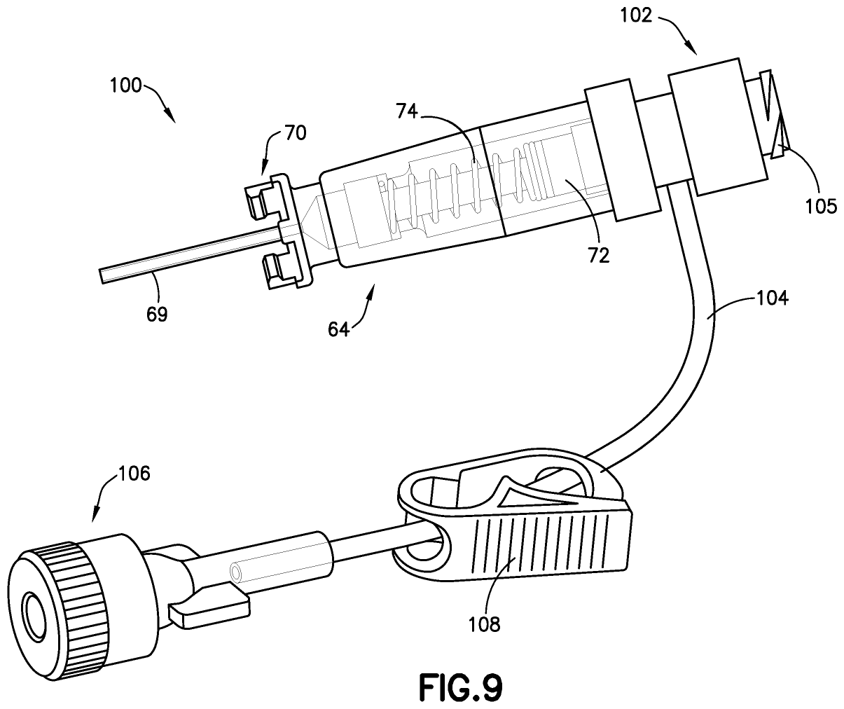

Referring now to FIG. 9, a needle-free connector assembly 100 in accordance with another aspect of the present disclosure is shown. Needle-free connector assembly 100 includes the connector assembly 64 described above with respect to FIGS. 6A and 6B, and further includes a T-extension set 102 coupled to a proximal end of the connector assembly 64. The T-extension set 102 comprises a proximal connector portion 105 capable of receiving, e.g., an elongated core of a blood draw device such as, e.g., a PIVO™ device. Similar to the needle-free connector assemblies described above with respect to FIGS. 7A-8, the T-extension set 102 is configured such that the elongated core of the blood draw device passes therethrough to engage the actuator 72 and provide fluid communication with the cannula 69. The T-extension set 102 also includes an extension tube 104 extending from a side surface thereof, with a proximal end of the extension tube 104 being coupled to a connector 106. In some embodiments, a clamp 108 may be provided over the extension tube 104 so as to allow for selective occlusion of fluid flow through the extension tube 104. The extension tube 104 and connector 106 allow for peripheral fluid injection (e.g., flushing fluid injection) into the T-extension set 102 and connector assembly 64.

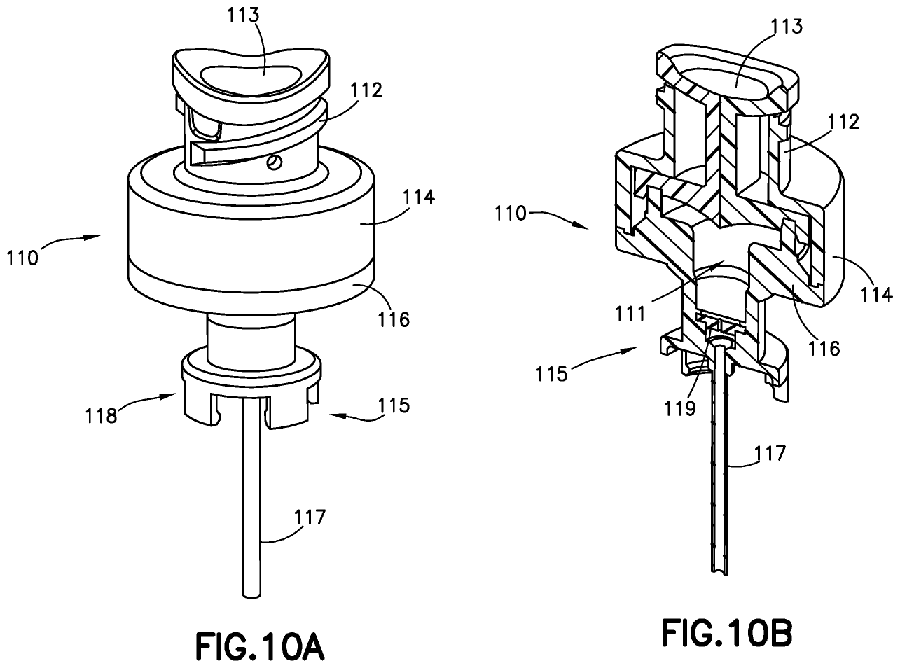

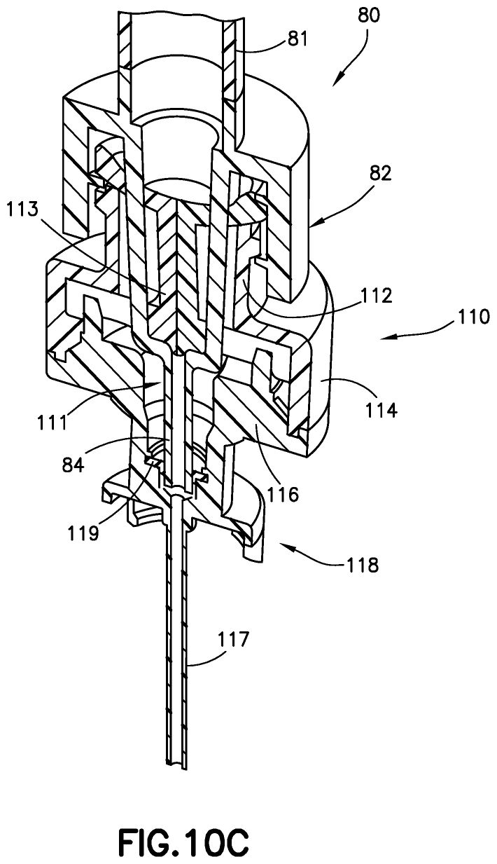

Next, referring to FIGS. 10A-10C, a needle-free connector assembly 110 in accordance with another aspect of the present disclosure is shown. Unlike the connector assemblies described herein with respect to FIGS. 1-9, needle-free connector assembly 110 does not utilize a sliding actuator or sliding cannula to selectively form a fluid path between a catheter adapter and a blood draw device. The needle-free connector assembly 110 includes a connector body 115, with connector body 115 having a coupling interface 118 and fixed cannula 117 extending distally therefrom. While not shown, it is to be understood that the coupling interface 118 is configured to secure the needle-free connector assembly 110 to a proximal portion of a catheter adapter, with the fixed cannula 117 sized and configured to pass through a septum configuration of the proximal portion of the catheter adapter so as to form a passage between the indwelling catheter and the needle-free connector assembly 110. A proximal portion 116 of the connector body 115 is coupled to a coupling portion 114. In some embodiments, the coupling portion 114 may be configured as, e.g., a BD Q-Syte™ Needle-Free Connector from Becton, Dickinson and Co. The coupling portion 114 may include a proximal interface portion 112, with the proximal interface portion 112 configured as a female luer connector having a split septum seal 113 formed therein.

As shown in FIG. 10C, when a blood draw device 80 is coupled to the needle-free connector assembly 110, the elongated core 84 of the blood draw device is configured to open the split septum seal 113 and pass through an interior channel 111 of the connector body 115. Additionally, a distal tip portion of the elongated core 84 is also configured to pass through a pre-punctured septum 119 positioned within the interior channel 111, thereby providing a conduit between the fixed cannula 117 and the blood draw device 80. In this way, a tube or probe (not shown) of the blood draw device 80 can be advanced through the needle-free connector assembly 110, the catheter adapter (not shown), and the indwelling catheter (not shown) to allow for blood collection. After the blood collection procedure, the blood draw device 80 can be disconnected from the proximal interface portion 112, thereby retracting the elongated core 84 such that both the pre-punctured septum 119 and the split septum seal 113 may close until another blood draw device is coupled thereto.

The needle-free connector assembly 110 is configured to provide an effective seal against blood reflux when a blood draw device is not coupled thereto. Additionally, the needle-free connector assembly 110 is disinfectable prior to initial use and between each use to aid in the prevention of sample contamination and/or the introduction of contaminants, and may be primed after connection to a catheter adapter.

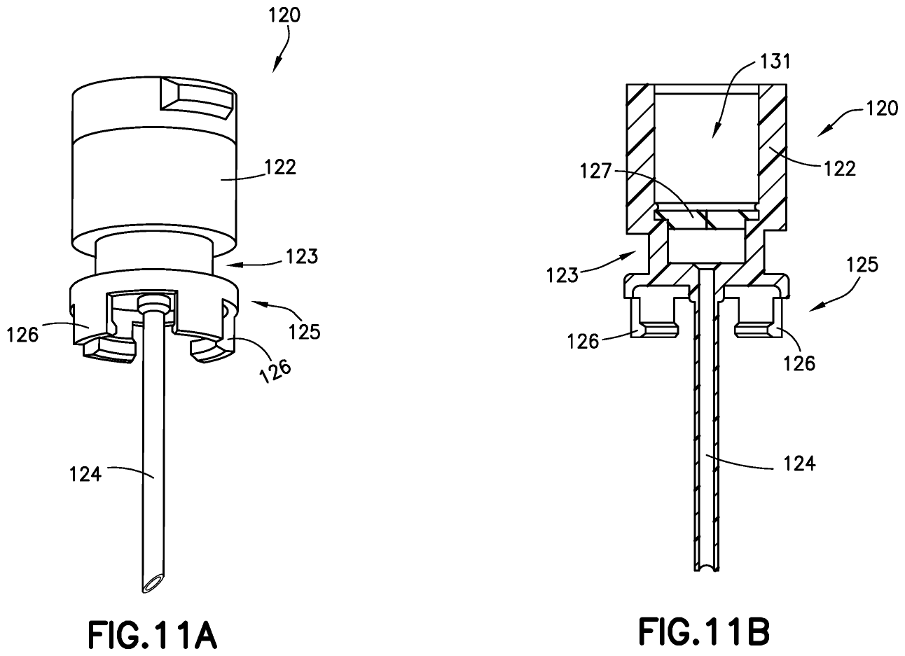

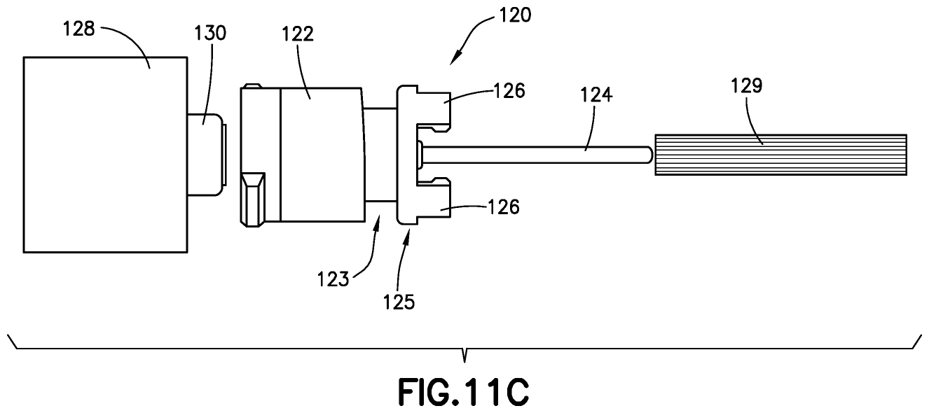

Referring now to FIGS. 11A-11C, a connector assembly 120 in accordance with another aspect of the present disclosure is shown. Similar to needle-free connector assembly 110 described above with respect to FIGS. 10A-10C, connector assembly 120 does not utilize a sliding actuator or sliding cannula to selectively form a channel between a catheter adapter and a blood draw device. The connector assembly 120 includes a connector body 122, with connector body 122 having a coupling interface 125 for coupling the connector assembly 120 to a proximal portion of a catheter adapter (not shown). In some embodiments, the coupling interface 125 may include a plurality of extensions 126, wherein the extensions 126 are configured to snap into a groove or recess formed on an outer surface of a proximal portion of the catheter adapter (not shown), thereby securing the connector assembly 120 to the catheter adapter, as well as preventing rotation of the connector assembly 120 with respect to the catheter adapter.

Connector assembly 120 further includes a fixed cannula 124 extending distally therefrom. While not shown, it is to be understood that the coupling interface 125 is configured to secure the connector assembly 120 to a proximal portion of a catheter adapter, with the fixed cannula 124 sized and configured to pass through a septum configuration of the proximal portion of the catheter adapter so as to form a passage between the indwelling catheter and the connector assembly 120. Rather than a split septum seal or other sealing feature, the connector assembly 120 may instead utilize a removable end cap 128, wherein an internal portion 130 of the end cap 128 is configured to selectively seal an interior channel 131 of the connector body 122.

When a blood draw device (not shown) is to be coupled to the connector assembly 120, the end cap 128 may be removed, thereby exposing the interior channel 131 of the connector body. The blood draw device may then be coupled to the connector assembly via, e.g., clip engagement with a groove 123 formed on the connector body 122. An elongated core of the blood draw device is configured to pass through the interior channel 131 of the connector body 122, with a distal tip portion of the elongated core also capable of passing through a pre-punctured septum 127 positioned within the interior channel 131, thereby providing a conduit between the fixed cannula 124 and the blood draw device. In this way, a tube or probe (not shown) of the blood draw device can be advanced through the connector assembly 120, the catheter adapter (not shown), and the indwelling catheter (not shown) to allow for blood collection. After the blood collection procedure, the blood draw device can be disconnected from the connector body 122, thereby retracting the elongated core such that the pre-punctured septum 127 may close until another blood draw device is coupled thereto. The end cap 128 may be repositioned on the connector body 122 between blood draw procedures. Additionally and/or alternatively, a cannula cover 129 may be provided to cover and protect the fixed cannula 124 prior to insertion into a catheter adapter.

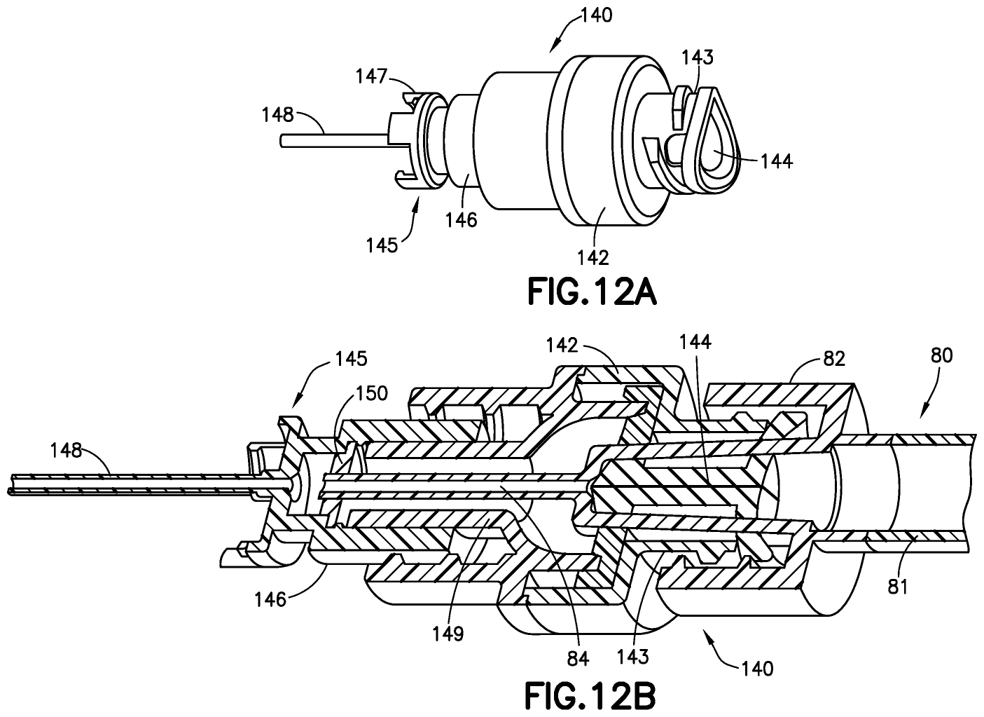

Next, referring to FIGS. 12A and 12B, a connector assembly 140 in accordance with another aspect of the present disclosure is illustrated. Similar to needle-free connector assembly 110 described above with respect to FIGS. 10A-10C, connector assembly 140 does not utilize a sliding actuator or sliding cannula to selectively form a channel between a catheter adapter and a blood draw device. The connector assembly 140 includes a connector body 146, with connector body 146 having a coupling interface 145 for coupling the connector assembly 140 to a proximal portion of a catheter adapter (not shown). In some embodiments, the coupling interface 145 may include a plurality of extensions 147, wherein the extensions 147 are configured to snap into a groove or recess formed on an outer surface of a proximal portion of the catheter adapter (not shown), thereby securing the connector assembly 140 to the catheter adapter, as well as preventing rotation of the connector assembly 140 with respect to the catheter adapter.

Connector assembly 140 further includes a fixed cannula 148 extending distally therefrom. While not shown, it is to be understood that the coupling interface 145 is configured to secure the connector assembly 140 to a proximal portion of a catheter adapter, with the fixed cannula 148 sized and configured to pass through a septum configuration of the proximal portion of the catheter adapter so as to form a passage between the indwelling catheter and the connector assembly 140.

Additionally, connector assembly 140 includes a needle-free connector 142, with the needle-free connector capable of being removably coupled to the connector body 146. In some embodiments, needle-free connector 142 may be configured as, e.g., a BD Q-Syte™ Needle-Free Connector from Becton, Dickinson and Co. However, it is to be understood that any appropriate needle-free connector may be used, such as, e.g., a BD SmartSite™ Needle-Free Connector from Becton, Dickinson and Co. The needle-free connector 142 may a proximal interface portion 143, with the proximal interface portion 143 being configured as a female luer connector with a split septum seal 144 formed therein.

Referring to FIG. 12B, the needle-free connector 142 is configured for use with the blood draw device 80, wherein the blood draw device 80 includes the male luer lock coupling member 82 and the elongated core 84 extending distally from the male luer lock coupling member 82. As shown in FIG. 12B, when the blood draw device 80 is coupled to the needle-free connector 142, the elongated core 84 is configured to open the split septum seal 144 and pass through a male luer slip 149 of a distal interface portion. Additionally, a distal tip portion of the elongated core 84 is also configured to pass through a pre-punctured septum 150 positioned within an interior channel of the connector body, thereby providing a conduit between the fixed cannula 148 and the blood draw device 80. In this way, a tube or probe (not shown) of the blood draw device 80 can be advanced through the connector assembly 140, the catheter adapter (not shown), and the indwelling catheter (not shown) to allow for blood collection. After the blood collection procedure, the blood draw device 80 can be disconnected from the proximal interface portion 143, thereby retracting the elongated core 84 such that both the pre-punctured septum 150 and the split septum seal 144 may close until another blood draw device is coupled thereto.

The connector assembly 140 is configured to provide an effective seal against blood reflux when a blood draw device is not coupled thereto. Additionally, the connector assembly 140 is disinfectable prior to initial use and between each use to aid in the prevention of sample contamination and/or the introduction of contaminants.

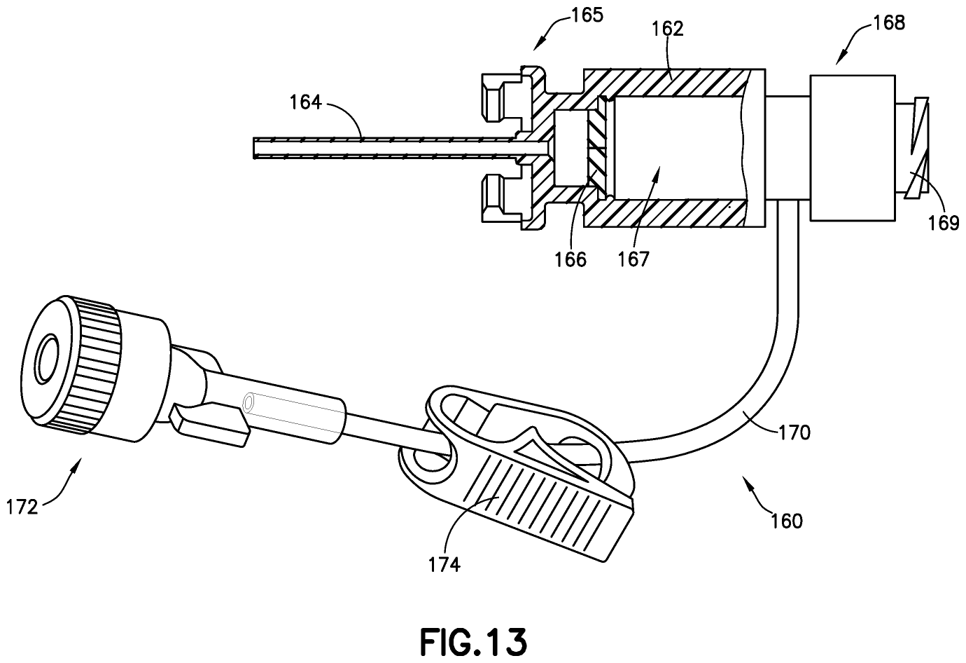

Referring now to FIG. 13, a needle-free connector assembly 160 in accordance with another aspect of the present disclosure is shown. Needle-free connector assembly 160 includes a connector body 162, with connector body 162 having a coupling interface 165 for coupling the connector assembly 162 to a proximal portion of a catheter adapter (not shown). Connector assembly 162 further includes a fixed cannula 164 extending distally therefrom. While not shown, it is to be understood that the coupling interface 165 is configured to secure the needle-free connector assembly 160 to a proximal portion of a catheter adapter, with the fixed cannula 164 sized and configured to pass through a septum configuration of the proximal portion of the catheter adapter so as to form a passage between the indwelling catheter and the needle-free connector assembly 160.

The needle-free connector assembly 160 further includes a T-extension set 168 coupled to a proximal end of the connector body 162. The T-extension set 168 comprises a proximal connector portion 169 capable of receiving, e.g., an elongated core of a blood draw device such as, e.g., a PIVO™ device. The T-extension set 168 is configured such that the elongated core of the blood draw device passes therethrough enter an interior channel 167 formed within the connector body, with at least a distal end portion of the elongated core also passing through a pre-punctured septum 166 positioned within the interior channel, thereby providing fluid communication with the fixed cannula 164. When the blood draw device is disconnected from the T-extension set 168, the elongated core is withdrawn such that the septum 166 re-seals, thereby acting as an anti-reflux valve to shield a distal end of the catheter (not shown) from experiencing any potential reflux generated from the needle-free configuration of the proximal connector portion 169.

The T-extension set 168 also includes an extension tube 170 extending from a side surface thereof, with a proximal end of the extension tube 170 being coupled to a connector 172. In some embodiments, a clamp 174 may be provided over the extension tube 170 so as to allow for selective occlusion of fluid flow through the extension tube 170. The extension tube 170 and connector 172 allow for peripheral fluid injection (e.g., flushing fluid injection) into the T-extension set 168 and the connector body 162.

While several embodiments of connectors for use in conjunction with catheter adapters and blood draw devices for blood draw procedures during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

We claim:

1. A vascular access system configured for delivery of a tube or probe into a patient's vascular system, the vascular access system comprising:
a catheter adapter comprising a distal portion and a proximal portion, wherein a catheter extends from the distal portion and a septum assembly is housed within the proximal portion; and
a connector assembly, wherein the connector assembly comprises:
a coupling interface configured to couple the connector assembly to the proximal portion of the catheter adapter,
a cannula, wherein the cannula is configured to extend through the septum assembly of the catheter adapter when the connector assembly is coupled to the proximal portion of the catheter adapter; and
a proximal interface, wherein the proximal interface is configured for coupling to a blood draw device having an elongated core extending from a distal end thereof.

2. The vascular access system of claim 1, wherein at least a portion of the cannula is slidable within the connector assembly.

3. The vascular access system of claim 2, wherein the connector assembly further comprises a biasing member, and wherein the biasing member is configured to bias the cannula proximally within the connector assembly.

4. The vascular access system of claim 3, wherein the biasing member is a steel spring.

5. The vascular access system of claim 3, wherein the biasing member is a rubber bellow.

6. The vascular access system of claim 2, wherein the cannula is configured to at least partially extend through an opening formed in a distal end portion of the connector assembly.

7. The vascular access system of claim 6, further comprising an O-ring seal surrounding the opening formed in the distal end portion of the connector assembly.

8. The vascular access system of claim 2, further comprising a split septum positioned at a proximal end portion of the connector assembly.

9. The vascular access system of claim 2, wherein the cannula comprises a proximal interface surface, and wherein the proximal interface surface is configured to receive at least a portion of a distal end of the elongated core of the blood draw device when the blood draw device is coupled to the connector assembly.

10. The vascular access system of claim 1, wherein the coupling interface of the connector assembly comprises a plurality of extensions projecting distally therefrom.

11. The vascular access system of claim 1, wherein the connector assembly further comprises a groove formed in a sidewall thereof, and wherein the groove is configured to receive a distal end of one or more clips of a coupler member of the blood draw device.

12. The vascular access system of claim 1, wherein the cannula is fixed and extends distally from the coupling interface of the connector assembly.

13. The vascular access system of claim 12, wherein the connector assembly further comprises a slidable actuator disposed therein, and wherein the actuator is configured to selectively pierce a split septum positioned within a body of the connector assembly.

14. The vascular access system of claim 1, wherein the proximal interface of the connector assembly comprises a needle-free connector.

15. The vascular access system of claim 1, wherein the proximal interface of the connector assembly comprises a T-extension set.

16. A connector assembly configured for use with a vascular access system for delivery of a tube or probe into a patient's vascular system, the connector assembly comprising:
a coupling interface configured to couple the connector assembly to a proximal portion of a catheter adapter of the vascular access system;
a cannula, wherein the cannula is configured to selectively extend through a septum assembly of the catheter adapter; and
a proximal interface, wherein the proximal interface is configured for coupling to a blood draw device having an elongated core extending from a distal end thereof.

17. The connector assembly of claim 16, wherein at least a portion of the cannula is slidable within the connector assembly.

18. The connector assembly of claim 17, further comprising a biasing member, wherein the biasing member is configured to bias the cannula proximally within the connector assembly.

19. The connector assembly of claim 16, wherein the cannula is fixed and extends distally from the coupling interface of the connector assembly.

20. The connector assembly of claim 19, further comprising a slidable actuator disposed therein, wherein the actuator is configured to selectively pierce a split septum positioned within a body of the connector assembly.
